(19) United States
(12) Patent Application Publication
(10) Pub. No.: US 2025/0034464 A1
(43) Pub. Date: Jan. 30, 2025

(54) METHOD AND DEVICE FOR SEPARATING A HYDROCARBON-CONTAINING FEEDSTOCK STREAM BY EXTRACTIVE DISTILLATION

(71) Applicants: thyssenkrupp Uhde GmbH, Dortmund (DE); thyssenkrupp Uhde Engineering Services GmbH, Bad Soden am Taunus (DE); thyssenkrupp AG, Essen (DE)

(72) Inventors: Michael KLEIBER, Hattersheim (DE); Simone BETTINGER, Bad Soden/Taunus (DE); Frank SCHUCH, Dortmund (DE); Martin GRATOWSKI, Frankfurt (DE); Helmut GEHRKE, Bergkamen (DE)

(73) Assignees: thyssenkrupp Uhde GmbH, Dortmund (DE); thyssenkrupp Uhde Engineering Services GmbH, Bad Soden am Taunus (DE); thyssenkrupp AG, Essen (DE)

(21) Appl. No.: 18/709,457

(22) PCT Filed: Nov. 2, 2022
(86) PCT No.: PCT/EP2022/080504

§ 371 Date: May 10, 2024

(30) Foreign Application Priority Data
Nov. 12, 2021 (BE) BE 2021/5879
Nov. 12, 2021 (DE) 10 2021 212 776.2

(51) Int. Cl. C10G 53/04; B01D 3/14; B01D 3/40; B01D 11/04

(52) U.S. Cl. CPC C10G 53/04; B01D 3/143; B01D 3/40; B01D 11/0446; B01D 11/0488; C10G 2400/30

(57) ABSTRACT

The disclosure relates to a process for separating a hydrocarbon-containing feedstock stream by extractive distillation. The feedstock stream is contacted with a water-soluble solvent for aromatics in countercurrent, an aliphatics fraction is distillatively removed as aliphatics product stream from the mixture obtained. The aromatics are stripped from the aromatics-enriched solvent that remains and the aromatics-depleted solvent is recycled in a solvent circuit, with aliphatic compounds and/or compounds with aliphatic moieties accumulating in the solvent circuit as impurities. At least a substream of the solvent circuit is purified for removal of the impurities, wherein for the purifying, a liquid/liquid extraction is carried out between the substream and an extractant for aliphatics and the raffinate of the liquid/liquid extraction is recycled into the solvent circuit. An apparatus for carrying out the process is also disclosed.

### PRIOR ART

The invention relates to a process for separating a hydrocarbon-containing feedstock stream by extractive distillation according to the preamble of claim 1 and to an apparatus according to the preamble of claim 10.

Aromatics, in particular the simplest aromatic compounds benzene, toluene and xylene, are of industrial importance as intermediates for the chemical industry. Various industrial processes are known for obtaining aromatics. One type of process which achieves a particularly high purity of the aromatics product stream with comparatively low costs is the obtaining by extractive distillation from a hydrocarbon-containing feedstock stream. Examples of feedstock streams that may be considered include naphtha, pyrolysis gasoline, reformate gasoline or also coking plant light oil. Heavy components are preferably removed from the feedstock stream prior to recovery of the aromatics, for example by removal of the C₈₊ fraction.

When recovering aromatics by extractive distillation, the feedstock stream is contacted in countercurrent with a selective solvent for aromatics. The solvent influences the volatility of the various constituents of the feedstock stream to varying degrees. The volatility of the aromatic constituents is reduced by the dilution, while that of the aliphatic constituents is markedly increased. In this way, a distillative separation into aromatics and nonaromatics/aliphatics is made possible. In a first step, the aromatics dissolved in the solvent are then separated by means of extractive distillation from the aliphatic constituents of the feed mixture, which are discharged as top product of the distillation. In a second step, the aromatics are driven out from the solvent as top product by stripping. The aromatics product stream can then be separated further into individual aromatics fractions. The aromatics-depleted solvent is recycled into the extractive distillation and reused. In this way, individual aromatics can be obtained in pure form in a continuous process.

Solvents known for the extractive distillation include, inter alia, sulfolane, methylsulfolanes, N-methylpyrrolidone, N-formylmorpholine, ethylene glycol and mixtures thereof, and also mixtures of the mentioned solvents with water. The solvents or solvent mixtures used are water-soluble.

The sustained reuse of the same solvent in a solvent circuit results in the accumulation in the solvent of impurities, which neither leave the process as top product of the distillative removal of the aliphatics nor as top product in the stripping of the aromatics. As a result of operator efforts to optimize the energy efficiency of the plants and to save operating materials, such impurities arise to an increasing extent also in the feedstock stream of plants serving solely to recover benzene and toluene. Especially in plants that are used not just for the recovery of benzene and toluene but also at the same time for the recovery of xylene, the accumulation of such impurities can hardly be avoided over the long term, since the feedstock stream in this case must contain a relatively large proportion of higher-boiling components. In addition, processes connected upstream of the extractive distillation, such as for example clay treatment, can introduce heavy components that accumulate in the solvent. However, it is also possible that, as a result of incorrect operation or malfunctions of plant parts connected upstream of the extractive distillation, these parts for example serving to remove higher-boiling fractions, there is increased contamination of the solvent circulating in the extractive distillation with high-boiling impurities.

Over the course of time, the impurities lead to the solvent losing its extractant power and needing to be replaced. In order to reduce the costs associated with replacing the solvent, it is desirable to have the option of purifying the solvent in the solvent circuit, by means of which the impurities are removed. In this way, the cycle time until replacement of the solvent can be considerably extended.

For purification of the solvent, it is known for example from US 2010/0228072 A1 to subject a substream of the solvent circuit to a distillation, with the purified solvent leaving the distillation as top product and impurities remaining behind as distillation residue being removed from the plant. The disadvantage with this type of purification is that only impurities that are higher boiling compared to the solvent can be removed from the solvent circuit. Impurities that are co-boilers with the solvent, that is to say which have a very similar boiling point, remain in the solvent.

It is thus known from DE 10 2012 111 292 A1 to add water to a substream of the extractant withdrawn from the bottom of the stripper column and supply this substream to a distillation column. In the distillation column, the extractant is separated from the added water and from hydrocarbons dissolved in the extractant. Water and hydrocarbons are discharged via the top of the distillation column. This type of purification is based on the extractant dissolving in the water and the water-insoluble hydrocarbons thus being displaced from solution in the extractant and forming a more volatile phase that can be removed with the water by distillation. However, the disadvantage is that a large amount of water needs to be used in order to treat the entire extractant. The entire amount of water used, at least in the case of an extractive distillation with largely anhydrous solvent, must then be distilled off again. The known process for purifying the solvent is thus associated with high energy expenditure and costs.

### DISCLOSURE OF THE INVENTION

An object of the invention is thus to specify a process and an apparatus for separating a hydrocarbon-containing feedstock stream by extractive distillation, in which the selectivity and capacity of the solvent used is ensured over a long period by means of a resource-efficient process.

This object is achieved by a process for separating a hydrocarbon-containing feedstock stream by extractive distillation having the features of claim 1 and by an apparatus having the features of claim 10.

A process for separating a hydrocarbon-containing feedstock stream by extractive distillation into at least one aliphatics product stream and one aromatics product stream is provided thereby, comprising the following steps:
contacting the feedstock stream with a water-soluble solvent for aromatics in countercurrent,
distillatively removing an aliphatics fraction from the mixture obtained to leave behind the aromatics-enriched solvent and discharging the aliphatics fraction in the aliphatics product stream,
stripping the aromatics from the aromatics-enriched solvent and discharging the aromatics in the aromatics product stream,
recycling the aromatics-depleted solvent in a solvent circuit for the extraction of further aromatics from the feedstock stream, with aliphatic compounds and/or compounds with aliphatic moieties accumulating in the solvent circuit as impurities, and
purifying at least a substream of the aromatics-depleted solvent for removal of the impurities.

According to the invention, there is provision that, for the purifying, a liquid/liquid extraction is carried out between the substream and an extractant for aliphatics, with the raffinate of the liquid/liquid extraction being recycled into the solvent circuit.

For the purposes of the invention, the substream can comprise any desired proportion of the overall solvent circuit. In particular, the substream can also be the entire solvent stream in the solvent circuit. Preference is given to a substream in the purification that corresponds to a proportion of 1% by mass to 20% by mass of the overall solvent stream in the solvent circuit.

As a result of the liquid/liquid extraction with an extractant for aliphatics, the aliphatic impurities pass over from the solvent into the extractant. The extractant for aliphatics may moreover also be suitable for extracting other impurities, such as for example naphthalene. In addition to the extractant, the extract of the liquid/liquid extraction therefore also comprises the aliphatic impurities and is saturated with solvent. The raffinate of the liquid/liquid extraction is a purified solvent stream that is saturated with the extractant and can be reused for the extractive distillation. The ratio by mass of extractant for aliphatics to solvent is preferably 1/20 to 10/1, preferably 1/10 to 5/1 and particularly preferably 1/5 to 2/1.

Through the use of a liquid/liquid extraction for removal of the aliphatic impurities with an extractant for aliphatics, the impurities are thus removed from the solvent as solvate. In known processes for purification by means of addition of water, in contrast, the solvent itself passes over into the water as solvate, while the impurities form an aliphatic, water-insoluble phase. According to the invention, the solvent can thus be purified without the addition of water to the substream to be purified. This is particularly advantageous in processes for the direct, water-free production of aromatics, in which the water content in the solvent circuit has to be kept sufficiently low since a subsequent energy-intensive removal of the water can be dispensed with.

Preference is given to using an extractant for aliphatics which in the extractive distillation is discharged from the solvent circuit via the aliphatics fraction. Examples of such extractants that can be considered include alkanes, alkane or aliphatics mixtures, or else oxygenates, such as MTBE or ETBE. The extractant for aliphatics is preferably C5-C9 hydrocarbons, especially paraffins or isomers thereof. Blends of aromatics and cyclic nonaromatics (naphthenes) in the extractant are also conceivable. In particular, the extractant can be obtained by branching off a second substream from the aliphatics product stream.

Alternatively, it is conceivable to use an extractant for aliphatics the accumulation of which in the solvent circuit up to the saturation limit has only a tolerable adverse effect on the selectivity and capacity of the solvent.

In preferred embodiments, for the recovery of solvent from the extract of the liquid/liquid extraction, the following further steps are carried out:
adding water to the extract of the liquid/liquid extraction to form an aqueous solvent-containing phase and a hydrophobic phase,
separating the aqueous phase from the hydrophobic phase and
distilling the aqueous phase for the removal of water by distillation, with the bottom product of the distillation being recycled into the solvent circuit.

The ratio by mass of extract to water is preferably 1/20 to 40/1, preferably 1/10 to 30/1 and particularly preferably 1/5 to 20/1. The addition of water and the separation of the aqueous phase are preferably effected at a temperature in the range from 0° C. to 80° C. and a pressure from 0 bar to 40 bar positive pressure.

The recovery of the solvent from the solvent-saturated extract of the liquid/liquid extraction leads to a lower solvent consumption and facilitates the further processing/disposal of the extract. The recovery of the solvent from the extract thus constitutes a particularly cost-efficient and environmentally friendly variant of the process according to the invention.

If for the recovery of the solvent water is added only to the extract of the liquid/liquid extraction and not to the entire substream to be purified, the amount of water to be used is markedly reduced, since the extract has a solvent content that is lower by orders of magnitude. The energy requirement for the distillation of the aqueous phase is thus substantially reduced compared to the known processes for solvent purification that are based solely on washing with water.

Preferably, the water removed by distillation is recycled in a water circuit and again added to the extract of the liquid/liquid extraction. The reuse of the water in a water circuit reduces the amount of waste waters requiring treatment produced and ensures resource-efficient operation.

It is further preferable that the substream is cooled in a heat exchanger prior to the liquid/liquid extraction and the distillation is carried out using the heat energy obtained in the heat exchanger. For carrying out the liquid/liquid extraction, a temperature of the substream in the range from 0° C. to 80° C. is advantageous and a pressure of 0 to 40 bar positive pressure is preferred. Extraction at a lower temperature compared to the temperature of the stripping (approx. 160° C.-240° C.) is preferred since this reduces the solubility of the impurities and of the extractant for aliphatics in the solvent. Excessively low temperatures should be avoided, in particular to prevent solidification of the solvent. The amount of energy obtained when cooling the substream in the heat exchanger is generally more than enough for carrying out the distillation. Excess heat energy can be utilized for heating the solvent being recycled into the solvent circuit. Alternatively or in addition, heat energy from the main stream of the solvent circuit can be extracted for operating the distillation column.

Preferably, the distillation is carried out with a top pressure of less than 1 bar (a), especially preferably less than 500 mbar (a) and particularly preferably less than 100 mbar (a). When carrying out the distillation in a vacuum, the boiling point of the water is lowered and thus the temperature in the distillation bottoms is reduced. This makes it possible to reduce the amount of energy required for the distillation and avoids undesirable side reactions in the distillation bottoms. The vacuum is preferably set such that the bottom temperature is in a range up to at most 230° C., particularly preferably between 150° C. and 200° C.

After water has been added to the extract of the liquid/liquid extraction and the aqueous phase has been separated, a hydrophobic phase is present containing extractant and impurities. In preferred embodiments of the process, there is provision that a removal of the impurities from the hydrophobic phase is carried out. Removal of the impurities is desirable if the impurities are disruptive to downstream processes or to the product purity.

Removal of the impurities is advantageous in particular when the hydrophobic phase is recycled into the liquid/liquid extraction as extractant for aliphatics in an extractant circuit. However, removal of the impurities may also be desirable, depending on the product requirements, when the hydrophobic phase is supplied to the aliphatics product stream.

Both the liquid/liquid extraction for the extraction of the aliphatics from the solvent and the distillative separation of water and solvent can be operated continuously or batchwise.

The process according to the invention is particularly advantageously usable in the direct preparation of anhydrous aromatics. For the purposes of the present disclosure, “anhydrous” means that the water content of the aromatics fraction after the extractive distillation immediately satisfies the requirements placed on the pure product and no drying steps downstream of the extractive distillation need to be carried out. In particular, it is then not necessary to remove a separate water phase from the condensed aromatics fraction. In such processes, the solvent in the solvent circuit (bottom of the stripper column) typically has a water content of less than 1% by mass, preferably less than 0.5% by mass and particularly preferably less than 1000 ppm. Moreover, no steam is added to the stripper column in this variant either, since this would result in an increased water content in the aromatics fraction.

Solvents useful for the extractive distillation include, inter alia, sulfolane, methylsulfolanes, N-methylpyrrolidone, N-formylmorpholine, ethylene glycol and mixtures thereof, and also mixtures of the mentioned solvents with water. Particularly preferably, the solvent contains N-formylmorpholine, which is particularly suitable for the direct preparation of anhydrous aromatics.

In terms of apparatus, the object is achieved by an apparatus for separating a hydrocarbon-containing feedstock stream by extractive distillation into at least one aliphatics product stream and one aromatics product stream, comprising:
a device for contacting the feedstock stream with a water-soluble solvent for aromatics in countercurrent,
a device for distillatively removing an aliphatics fraction from the mixture obtained to leave behind the aromatics-enriched solvent, with a discharge for the aliphatics fraction as aliphatics product stream,
a device for stripping the aromatics from the aromatics-enriched solvent with a discharge for the aromatics as aromatics product stream,
a recycle line for the aromatics-depleted solvent in a solvent circuit to the device for contacting the feedstock stream with solvent, and
a purification device for the solvent, which is arranged in the solvent circuit and through which at least temporarily at least a substream of the aromatics-depleted solvent passes during operation, for the removal of impurities comprising aliphatic compounds and/or compounds with aliphatic moieties from the substream.

According to the invention, the purification device contains a feed for an extractant for aliphatics and at least one mixing chamber and one separating chamber for a liquid/liquid extraction between the substream and an extractant for aliphatics. The purification device further includes a recycle line for a raffinate of the liquid/liquid extraction into the solvent circuit.

In preferred embodiments, the purification apparatus includes a discharge for an extract of the liquid/liquid extraction, which is connected to a solvent recovery device. Arranged in the solvent recovery device are at least one mixing apparatus for the addition of water to form an aqueous phase and a hydrophobic phase and at least one separating apparatus for separating the aqueous phase from the hydrophobic phase. The solvent recovery device further includes a discharge for the aqueous phase which is connected to a distillation column for the removal of the water by distillation. The distillation column includes a discharge for a bottom product of the distillation column, via which the bottom product is recyclable into the solvent circuit.

The distillation column preferably includes a top discharge for the water removed by distillation, which is connected to the mixing apparatus for the addition of water to form a water circuit.

Furthermore, arranged upstream of the purification apparatus is preferably at least one heat exchanger for cooling the substream, which is connected to the distillation column for transfer of the heat energy obtained in the heat exchanger.

In preferred embodiments, the distillation column is connected to a vacuum generation device configured to generate a negative pressure of less than 1 bar (a), preferably less than 500 mbar (a) and particularly preferably less than 100 mbar (a) in a top region of the distillation column.

In further preferred embodiments, a separation device for removing the impurities from the hydrophobic phase is provided. The separation device may, for example, be a further distillation device, for example a distillation column. Preferably, the separation device is connected to the feed of the purification device to form an extractant circuit.

Further advantageous embodiments can be gathered from the following description and the dependent claims.

The invention is explained in more detail below with reference to the exemplary embodiments illustrated in the accompanying figures.

### BRIEF DESCRIPTION OF THE DRAWINGS

FIG. 1 schematically shows a flow diagram of the process according to the invention according to a first exemplary embodiment, in which a second substream is branched off from the aliphatics product stream as extractant for aliphatics and the hydrophobic phase is supplied to the aliphatics product stream,

FIG. 2 schematically shows a flow diagram of the process according to the invention according to a second exemplary embodiment, in which the impurities are removed from the extractant for aliphatics and the extractant is conducted in an extractant circuit,

FIG. 3 schematically shows a first exemplary embodiment of the apparatus according to the invention, which is suitable for performing the process shown in FIG. 1,

FIG. 4 schematically shows a second exemplary embodiment of the apparatus according to the invention, which is suitable for performing the process shown in FIG. 2,

FIG. 5 schematically shows an alternative apparatus construction to the section X in FIGS. 3 and 4, for carrying out the extractive distillation in a single column for extractive distillation,

FIG. 6 schematically shows an alternative apparatus construction to the section X in FIGS. 3 and 4, for carrying out the extractive distillation in three separate columns.

### EMBODIMENTS OF THE INVENTION

In the various figures, identical parts are always provided with the same reference signs and are therefore also generally named or mentioned only once.

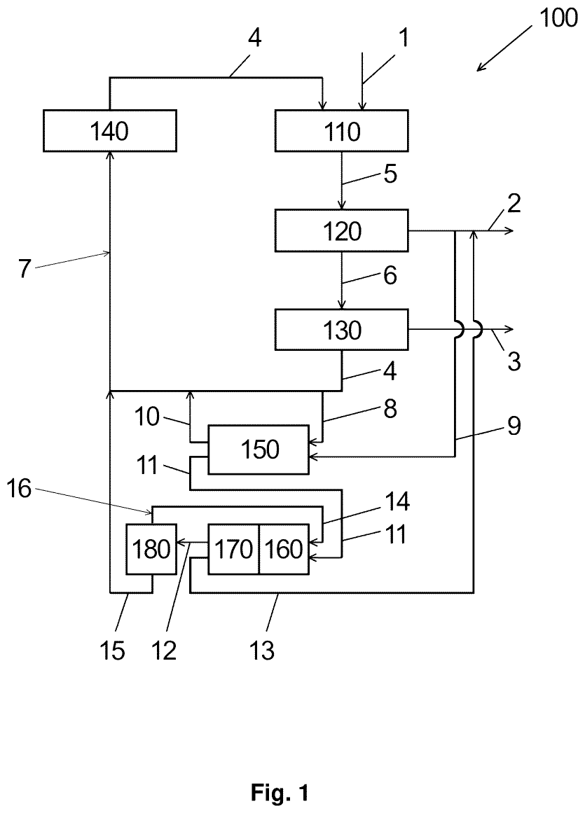

FIG. 1 shows a flow diagram of the process 100 according to the invention according to a first exemplary embodiment of the invention. In the process 100, a hydrocarbon-containing feedstock stream 1 is separated by extractive distillation into at least one aliphatics product stream 2 and one aromatics product stream 3.

Process 100 comprises the following steps:

In step 110, the feedstock stream 1 is contacted with a water-soluble solvent 4 for aromatics in countercurrent, and a mixture 5 is formed. Then, in step 120, an aliphatics fraction is distillatively removed from the mixture 5 obtained to leave behind the aromatics-enriched solvent 6 and the aliphatics fraction is discharged in the aliphatics product stream 2. Next, in step 130, the aromatics 130 are stripped from the aromatics-enriched solvent 6 and the aromatics are discharged in the aromatics product stream 3. The stripping is preferably effected at a higher temperature and/or lower pressure compared to the distillative removal of the aliphatics fraction. The aromatics-depleted solvent 4 is recycled in step 140 in a solvent circuit 7 for the extraction of further aromatics from the feedstock stream 1.

During the implementation of the process 100, aliphatic compounds and/or compounds with aliphatic moieties accumulate as impurities in the solvent circuit 7. Thus, in step 150, provision is made for purifying at least a substream 8 of the aromatics-depleted solvent 4 for removal of the impurities. For the purifying 150, a liquid/liquid extraction is carried out between the substream 8 and an extractant 9 for aliphatics, with the raffinate 10 of the liquid/liquid extraction being recycled into the solvent circuit 7. The substream 8 and the extractant 9 for aliphatics for the liquid/liquid extraction are preferably conducted in countercurrent to each other.

There may be provision for the purifying 150 of the solvent 4 to be effected only temporarily during the implementation of the process. To this end, the substream 8 may for example be controllable via a control valve. The volume flow rate of the substream 8 is preferably regulated such that the impurities in the solvent are adjusted to a target value or target range. The target value or target range for the impurities in the solvent is preferably in the range from 0.1-20% by mass, preferably 0.1-10% by mass and particularly preferably 1-5% by mass. In this way, the consumption of operating materials required for ensuring exceptional selectivity and capacity of the solvent can be reduced.

In the exemplary embodiment according to FIG. 1, a recovery of solvent from the extract 11 of the liquid/liquid extraction is further provided. For this purpose, the following further steps are carried out:

In step 160, water 14 is added to the extract 11 of the liquid/liquid extraction to form an aqueous solvent-containing phase 12 and a hydrophobic phase 13. Then, in step 170, the aqueous phase 12 is separated from the hydrophobic phase 13. Finally, in step 180, provision is made for distillation of the aqueous phase 12 for the removal of the water by distillation, with the bottom product 15 of the distillation 180 being recycled into the solvent circuit 7.

The distillation 180 is preferably carried out with a top pressure of less than 1 bar (a), more preferably less than 500 mbar (a) and particularly preferably less than 100 mbar (a). The bottom temperatures of the distillation 180 are preferably set in the range of less than 230° C., preferably in the range 150-200° C.

It is further preferably provided that the water 14 removed by distillation is recycled in a water circuit 16 and again added to the extract 11 of the liquid/liquid extraction. The hydrophobic phase 13 can, as shown in FIG. 1, be supplied to the aliphatics product stream 2. Prior to supply to the aliphatics product stream 2, the impurities may in some embodiments be removed from the hydrophobic phase. Alternatively, the hydrophobic phase 13 may also be discharged at the plant limits as a separate stream (not shown).

For the saving of energy it can preferably be provided that the substream 8 is cooled in a heat exchanger prior to the liquid/liquid extraction and the distillation is carried out using the heat energy obtained in the heat exchanger.

The solvent 4 in the solvent circuit 7 preferably has a water content of less than 3% by mass, more preferably less than 1% by mass and particularly preferably less than 1000 ppm.

In preferred process variants, the solvent 4, 6 contains N-formylmorpholine. Particularly preferably, the solvent 4 contains a proportion by mass of at least 50% N-formylmorpholine.

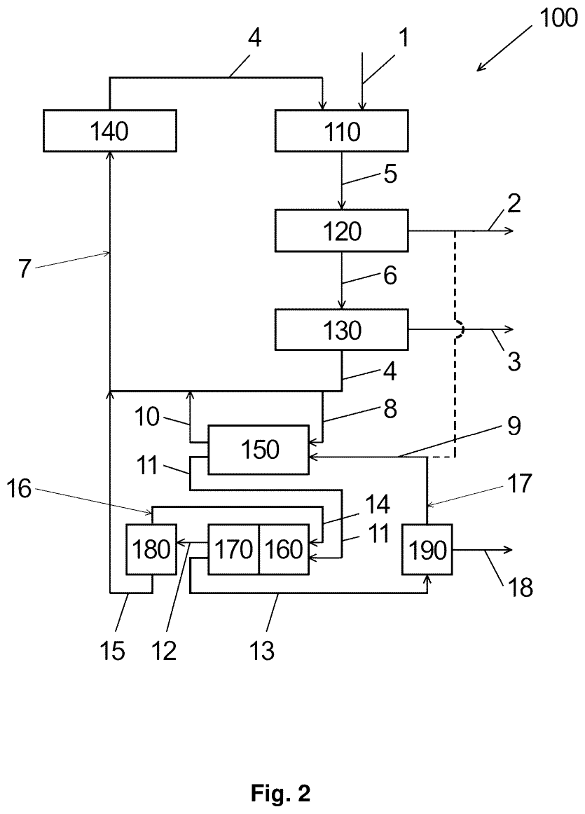

FIG. 2 shows a flow diagram of the process of the invention according to a second exemplary embodiment. The flow diagram differs from FIG. 1 only by the guiding of the extractant 9. In the second exemplary embodiment, the hydrophobic phase 13 is recycled into the liquid/liquid extraction as extractant 9 for aliphatics in an extractant circuit 17. The extractant is thus reused. In order to enable sustainable reuse of the extractant without replacement of the extractant, the second exemplary embodiment further provides for a removal 190 of the impurities from the hydrophobic phase 13 prior to recycling into the liquid/liquid extraction. The impurities 18 removed can be discharged separately and disposed of or be admixed with a stream the specification of which is not deleteriously impacted by the admixed impurities.

The extractant 9 can for example be obtained by discharge from the aliphatics product stream 2. Alternatively, other aliphatics may be used as extractant 9, for example alkanes, but also oxygenates such as MTBE and ETBE, which can be provided separately from the aliphatics product stream 2. Since a certain part of the extractant is always discharged from the extractant circuit 17 via the raffinate 12 and the bottom product 15, the extractant 9 is topped up from time to time.

For the remainder, the statements above in relation to the exemplary embodiment shown in FIG. 1 apply to FIG. 2, mutatis mutandis.

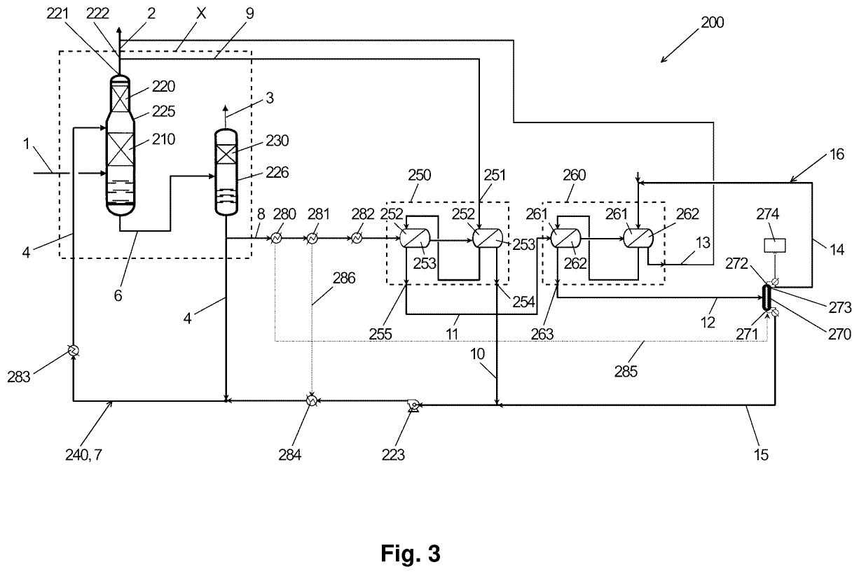

FIG. 3 shows a first exemplary embodiment of the apparatus according to the invention for separating a hydrocarbon-containing feedstock stream 1 by extractive distillation into at least one aliphatics product stream 2 and one aromatics product stream 3. The apparatus comprises a device 210 for contacting the feedstock stream with a water-soluble solvent 4 for aromatics in countercurrent, a device for distillatively removing an aliphatics fraction 220 from the mixture 5 obtained to leave behind the aromatics-enriched solvent 6, with a discharge 221 for the aliphatics fraction as aliphatics product stream 2, and a device for stripping 230 the aromatics from the aromatics-enriched solvent 6 with a discharge for the aromatics as aromatics product stream.

The apparatus further comprises a recycle line 240 for the aromatics-depleted solvent 4 in a solvent circuit 7 to the device 210 for contacting the feedstock stream 1 with solvent 4 in countercurrent, and a purification device 250 for the solvent 4, which is arranged in the solvent circuit 7 and through which at least a substream 8 of the aromatics-depleted solvent 4 passes during operation, for the removal of impurities comprising aliphatic compounds and/or compounds with aliphatic moieties from the substream 8.

The purification device 250 includes a feed 251 for an extractant 9 for aliphatics and at least one mixing chamber 252 and one separating chamber 253 for a liquid/liquid extraction between the substream 8 and the extractant 9 for aliphatics. The purification device 250 further includes a recycle line 254 for a raffinate 10 of the liquid/liquid extraction into the solvent circuit 7. The raffinate 10 can be supplied to the solvent circuit 7 for example via a pump 223.

In the exemplary embodiment illustrated in FIG. 3, the purification device 250 is equipped with a two-stage liquid/liquid extraction having two mixing chambers 252 and two separating chambers 253. In principle, a one-stage or also a more than two-stage liquid/liquid extraction are also usable. In multistage liquid/liquid extractions, extractant is added anew to the solvent in each stage in a mixing chamber 252 of the respective stage, and the solvent is separated again from the extractant in a separating chamber 253 of the respective stage. Preferably, solvent and extractant are conducted into the individual stages here in countercurrent. Particularly preferably used as the extractant of one stage is the extract from the following stage. The extract 11 of the liquid/liquid extraction is formed in such a multistage extraction by the extract of the first stage in the direction of flow of the solvent 8. In the case of a multistage liquid/liquid extraction, it is therefore possible to select a crosscurrent as well as a countercurrent process regime.

As illustrated in FIG. 3, the mixing chamber 252 and the separating chamber 253 may be formed within the same vessel, for example as a mixer-settler unit or else as a one-stage or multistage extraction column. The terms mixing chamber 252 and separating chamber 253 then denote different sections of the same cavity which, in operation, perform the respective function denoted by their name.

The purification apparatus 250 further includes a discharge 255 for the extract 11 of the liquid/liquid extraction, which is connected to a solvent recovery device 260. Arranged in the solvent recovery device 260 are at least one mixing apparatus 261 for the addition of water 14 to form an aqueous phase 12 and a hydrophobic phase 13 and at least one separating apparatus 262 for separating the aqueous phase 12 from the hydrophobic phase 13.

The solvent recovery device 260 may be of one-stage or multistage design. The mixing apparatus 261 may be formed together with the separating apparatus 262 in one vessel, for example a mixer-settler unit or else as a one-stage or multistage extraction column. However, the mixing apparatus 261 can also be formed merely by a pipeline branch via which water can be fed into the stream of the extract 11.

Preferably, extract 11 and water 14 are conducted in countercurrent in the solvent recovery device 260. Particularly preferably, in multistage recovery devices 260, the addition of water 14 is effected by adding the aqueous phase 12 from the respective following stage. In this way, the solvent accumulates over the stages in the aqueous phase in the opposite direction to the concentration of the solvent in the hydrophobic phase 13. The use of fresh or treated water can be reduced as a result.

The solvent recovery device 260 includes a discharge 263 for the aqueous phase 12 which is connected to a distillation column 270 for the removal of the water 14 by distillation. The distillation column 270 has a discharge 271 for a bottom product 15 of the distillation column 270, via which the bottom product 15 is recyclable into the solvent circuit 7.

The distillation column 270 further includes a top discharge 272 for the water 14 removed by distillation, which is connected to the mixing apparatus 261 for the addition of water 14 to form a water circuit 16.

The distillation column 270 is preferably connected to a vacuum generation device 274 configured to generate a negative pressure of less than 1 bar (a), preferably less than 500 mbar (a) and particularly preferably less than 100 mbar (a) in a top region 273 of the distillation column 270.

Preference is given to providing heat integration of the distillation column 270. To this end, arranged upstream of the purification apparatus 250 can be at least one heat exchanger 280 for cooling the substream 8, which is connected to the distillation column 270 for transfer of the heat energy 285 obtained in the heat exchanger 280. Particularly preferably, the entire energy requirement of the distillation column 270 is covered by the heat energy 285 transferred.

The cooling of the substream 8 prior to entry into the purification apparatus 250 is preferably effected down to a temperature range from 0° C. to 60° C. For this purpose, further heat exchangers 281, 282 may be provided, these preferably being arranged downstream of the heat exchanger 280 in the substream 8. The heat energy 286 additionally obtained in these heat exchangers 281, 282 can for example be used for heating the solvent stream made up of raffinate 10 and bottom product 15, before it is recycled into the solvent circuit 7. Alternatively or in addition, the heat energy may also be used at other points in the process of the extractive distillation.

After emerging from the solvent recovery device 260, the hydrophobic phase 13 can be discharged into the aliphatics product stream 2. A separation device for removing the impurities (not shown) from the hydrophobic phase 13 may be provided in the discharge. The separation device may for example be formed by a further distillation column. Alternatively to this, the impurities leave the apparatus 200 via the aliphatics product stream 2.

In the exemplary embodiment illustrated in FIG. 3, the main process of the extractive distillation is illustrated in the section X. Here, the device for contacting in countercurrent 210 and the device for distillative removal 220 are combined in a column for extractive distillation 225 and a separate stripper column 226 is provided for the device for stripping 230.

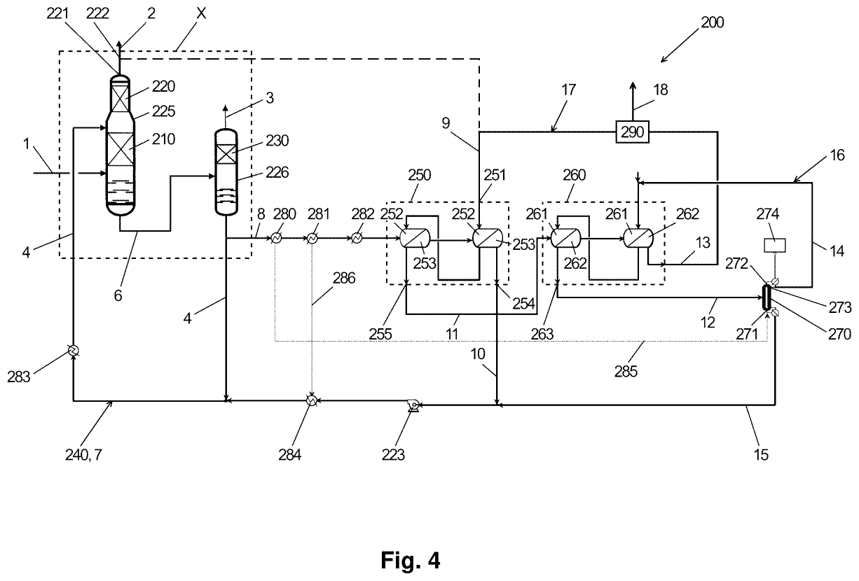

FIG. 4 shows a second exemplary embodiment of the apparatus according to the invention. The second exemplary embodiment differs from the first exemplary embodiment shown in FIG. 3 in that a separation device 290 for removing the impurities from the hydrophobic phase 13 is provided. The separation device 290 is connected to the feed 251 of the purification device 250 to form an extractant circuit 17. The impurities 18 removed can be discharged separately and disposed of or be admixed with a stream the specification of which is not deleteriously impacted by the admixed impurities.

For the remainder, the statements in relation to the first exemplary embodiment shown in FIG. 3 apply to FIG. 4, mutatis mutandis.

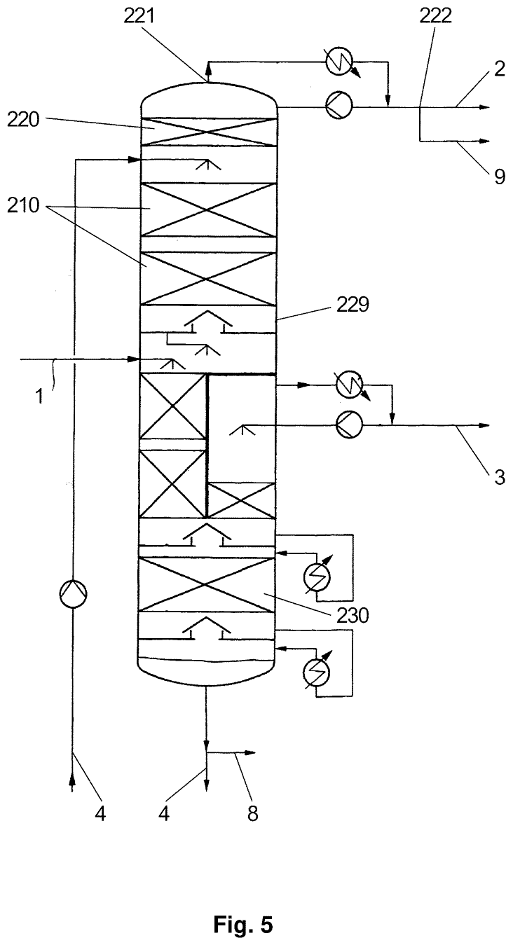

FIG. 5 shows an alternative apparatus construction for the section X in the apparatuses according to FIGS. 3 and 4. In the construction illustrated in FIG. 5, the device for contacting in countercurrent 210, the device for distillative removal 220 and the device for stripping 230 are combined in a single column for extractive distillation 229 with integrated stripping. The advantages of this construction are a lower apparatus expenditure and a lower space requirement for the apparatus 200.

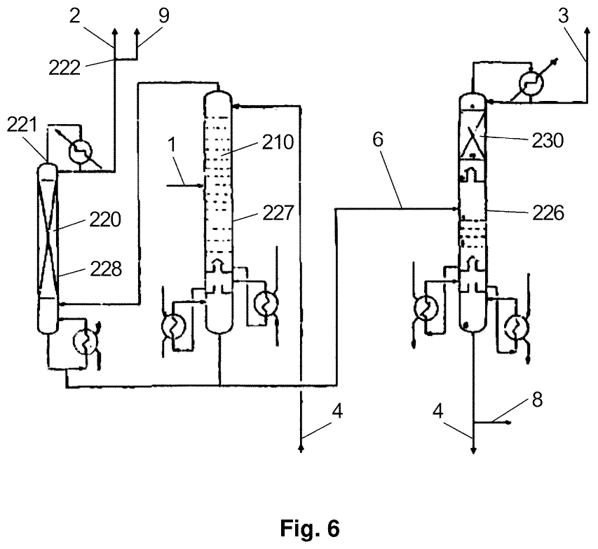

FIG. 6 shows a further alternative apparatus construction for the section X in the apparatuses according to FIGS. 3 and 4. In the construction illustrated in FIG. 6, a column for extractive distillation 227 is provided for the device for contacting in countercurrent 210 and a dedicated column for raffinate purification 228 is provided for the device for distillative removal 220. The device for stripping 230 is arranged in a separate stripper column 226. The advantages of this construction reside in the lower installation height for the column for extractive distillation 227. This construction can thus preferably be used in the case of practical or legal authorization restrictions in the installation height of the plant.

### LIST OF REFERENCE SIGNS

1 Feedstock stream
2 Aliphatics product stream
3 Aromatics product stream
4 Solvent for aromatics
5 Mixture of feedstock and solvent
6 Aromatics-enriched solvent
7 Solvent circuit
8 Substream of the solvent circuit
9 Extractant for aliphatics
10 Raffinate of the liquid/liquid extraction
11 Extract of the liquid/liquid extraction
12 Aqueous phase
13 Hydrophobic phase
14 Water
15 Bottom product of the distillation
16 Water circuit
17 Extractant circuit
18 Removed impurities
100 Process for separating a hydrocarbon-containing feedstock stream
110 Contacting of feedstock stream with solvent in countercurrent
120 Distillative removal of an aliphatics fraction
130 Stripping of the aromatics from the solvent
140 Recycling of the solvent
150 Purifying of the substream of the aromatics-depleted solvent
160 Adding of water
170 Separating of the aqueous phase from the hydrophobic phase
180 Distillation of the aqueous phase
190 Removal of impurities from the hydrophobic phase
200 Apparatus for separating a hydrocarbon-containing feedstock stream
210 Device for contacting in countercurrent
220 Device for distillative removal
221 Discharge
222 Branch-off
223 Pump
225, 227 Column for extractive distillation
226 Stripper column
228 Column for raffinate purification
229 Column for extractive distillation with integrated stripping
230 Device for stripping
240 Recycle line for the solvent
250 Purification device
251 Feed for the purification device
252 Mixing chamber
253 Separating chamber
254 Recycle line for raffinate
255 Discharge for extract
260 Solvent recovery device
261 Mixing apparatus
262 Separating apparatus
263 Discharge for the aqueous phase
270 Distillation column
271 Discharge for the bottom product
272 Top discharge
273 Top region
274 Vacuum generation device
280 to 284 Heat exchanger
285, 286 Heat energy
290 Separation device

1-16. (canceled)

17. A process for separating a hydrocarbon-containing feedstock stream by extractive distillation into at least one aliphatics product stream and one aromatics product stream, comprising the following steps:
contacting the feedstock stream with a water-soluble solvent for aromatics in countercurrent;
distillatively removing an aliphatics fraction from the mixture obtained to leave behind the aromatics-enriched solvent and discharging the aliphatics fraction in the aliphatics product stream;
stripping the aromatics from the aromatics-enriched solvent and discharging the aromatics in the aromatics product stream;
recycling the aromatics-depleted solvent in a solvent circuit for the extraction of further aromatics from the feedstock stream, with aliphatic compounds and/or compounds with aliphatic moieties accumulating in the solvent circuit as impurities; and
purifying at least a substream of the aromatics-depleted solvent for removal of the impurities, wherein for the purifying, a liquid/liquid extraction is carried out between the substream and an extractant for aliphatics, with the raffinate of the liquid/liquid extraction being recycled into the solvent circuit.

18. The process as claimed in claim 17, wherein, for the recovery of solvent from an extract of the liquid/liquid extraction, the following further steps are carried out:
adding water to the extract of the liquid/liquid extraction to form an aqueous solvent-containing phase and a hydrophobic phase;
separating the aqueous phase from the hydrophobic phase; and
distilling the aqueous phase for the removal of water by distillation, with the bottom product of the distillation being recycled into the solvent circuit.

19. The process as claimed in claim 18, wherein the water removed by distillation is recycled in a water circuit and again added to the extract of the liquid/liquid extraction.

20. The process as claimed in claim 18, wherein the substream is cooled in a heat exchanger prior to the liquid/liquid extraction and the distillation is carried out using the heat energy obtained in the heat exchanger.

21. The process as claimed in claim 18, wherein the distillation is carried out with a top pressure of less than 100 mbar (a).

22. The process as claimed in claim 18, wherein a removal of the impurities from the hydrophobic phase is carried out.

23. The process as claimed in claim 18, wherein the hydrophobic phase is one of (i) supplied to the aliphatics product stream; and (ii) recycled into the liquid/liquid extraction as extractant for aliphatics in an extractant circuit.

24. The process as claimed in claim 17, wherein the solvent in the solvent circuit has a water content of less than 1000 ppm.

25. The process as claimed in claim 17, wherein the solvent contains N-formylmorpholine.

26. An apparatus for separating a hydrocarbon-containing feedstock stream by extractive distillation into at least one aliphatics product stream and one aromatics product stream, comprising:
a device for contacting in countercurrent the feedstock stream with a water-soluble solvent for aromatics,
a device for distillatively removing an aliphatics fraction from the mixture obtained to leave behind the aromatics-enriched solvent, with a discharge for the aliphatics fraction as aliphatics product stream;
a device for stripping the aromatics from the aromatics-enriched solvent with a discharge for the aromatics as aromatics product stream;
a recycle line for the aromatics-depleted solvent in a solvent circuit to the device for contacting in countercurrent the feedstock stream with solvent; and
a purification device for the solvent, which is arranged in the solvent circuit and through which at least temporarily at least a substream of the aromatics-depleted solvent passes during operation, for the removal of impurities comprising aliphatic compounds and/or compounds with aliphatic moieties from the substream, wherein the purification device contains a feed for an extractant for aliphatics and at least one mixing chamber and one separating chamber for a liquid/liquid extraction between the substream and the extractant for aliphatics, and the purification device includes a recycle line for a raffinate of the liquid/liquid extraction into the solvent circuit.

27. The apparatus as claimed in claim 26, wherein the purification apparatus includes a discharge for an extract of the liquid/liquid extraction, which is connected to a solvent recovery device in which at least one mixing apparatus for the addition of water to form an aqueous phase and a hydrophobic phase and at least one separating apparatus for separating the aqueous phase from the hydrophobic phase are arranged, wherein the solvent recovery device includes a discharge for the aqueous phase which is connected to a distillation column for the removal of the water by distillation and the distillation column includes a discharge for a bottom product of the distillation column, via which the bottom product is recyclable into the solvent circuit.

28. The apparatus as claimed in claim 27, wherein the distillation column includes a top discharge for the water removed by distillation, which is connected to the mixing apparatus for the addition of water to form a water circuit.

29. The apparatus as claimed in claim 27, wherein arranged upstream of the purification apparatus is at least one heat exchanger for cooling the substream, which is connected to the distillation column for transfer of the heat energy obtained in the heat exchanger.

30. The apparatus as claimed in claim 27, wherein the distillation column is connected to a vacuum generation device configured to generate a negative pressure of less than 100 mbar (a) in a top region of the distillation column.

31. The apparatus as claimed in claim 27, wherein a separation device for removing the impurities from the hydrophobic phase is provided.

32. The apparatus as claimed in claim 31, wherein the separation device is connected to the feed of the purification device to form an extractant circuit.
